(19) United States
(12) Patent Application Publication
(10) Pub. No.: US 2019/0218341 A1
(43) Pub. Date: Jul. 18, 2019

(54) METHOD FOR THE PREPARATION OF POLYAMINES FROM DINITRILES AND/OR AMINO NITRILES

(71) Applicant: BASF SE, Ludwigshafen am Rhein (DE)

(72) Inventors: Thomas REISSNER, Ludwigshafen (DE); Ansgar Gereon ALTENHOFF, Ludwigshafen (DE); Christian MUELLER, Ludwigshafen (DE); Rolf-Hartmuth FISCHER, (US)

(73) Assignee: BASF SE, Ludwigshafen am Rhein (DE)

(21) Appl. No.: 16/331,440

(22) PCT Filed: Sep. 8, 2017
(86) PCT No.: PCT/EP2017/072602

§ 371 Date: Mar. 7, 2019

(30) Foreign Application Priority Data
Sep. 19, 2016 (EP) 16189422.5

(51) Int. Cl. C08G 73/02

(52) U.S. Cl. CPC C08G 73/0213; C07C 209/48; C08G 73/026

(57) ABSTRACT

The present invention relates to a process for one-stage preparation of polyamines by reacting compounds comprising at least two nitrile groups (dinitrile) or at least one nitrile group and one amino group (amino nitrile) with hydrogen in the presence of heterogeneous transition metal catalysts.

The present invention relates to a process for one-stage preparation of polyamines by reacting compounds comprising at least two nitrile groups (dinitrile) or at least one nitrile group and one amino group (amino nitrile) with hydrogen in the presence of heterogeneous transition metal catalysts.

Polyamines are oligomers or polymers having repeat chain units of the formula R—NH—R′ or R—NR″—R′. Molecules that can be distillatively removed by distillation overhead up to a pressure of 1 mbar are referred to in this application as oligomers. These oligomers are recyclable into the synthesis and consist of 2 to 4 repeat units of the monomer. Molecules that cannot be separated off by distillation in this way are referred to as polymers.

Polyamines can be prepared, for example, proceeding from cyclic imines, such as aziridines, cyclic imino ethers, such as oxazolines, and amino alcohols as described in WO 2014/131649 on pages 3 and 5.

The most versatile process for preparing polyamines is the polycondensation of diamines in the presence of catalysts based on transition metals, which is described in WO2014/131649 and the literature cited therein. By continuously separating off the ammonia formed during the polycondensation with flowing hydrogen, it is possible according to WO 2014/131649 to distinctly increase diamine conversion, polyamine yield and average molar mass.

The diamines required as starting compounds for the preparation of polyamines can be prepared by catalytic reductive amination of diols, dialdehydes or diketones with ammonia and hydrogen or preferably by catalytic hydrogenation of dinitriles or amino nitriles (S. Gomez, J. A. Peters, Th. Maschmeyer, Adv. Synth. Catal. 2002, 344, pages 1037 to 1057 and C. de Bellefon, P. Fouilloux, Catal. Rev. Sci. Eng., 36(3), pages 459 to 506).

For the processes known from the prior art for preparing diamines and/or amino nitriles as starting compounds for the preparation of polyamines, a large number of reaction steps is often necessary. This can lead to considerable capital costs and production costs.

For example, adiponitrile (ADN) is first prepared in a multistage process proceeding from butadiene and hydrogen cyanide, then catalytically hydrogenated in the presence of nickel catalysts, cobalt catalysts or iron catalysts to give hexamethylenediamine (HMD) as described by Hans-Jürgen Arpe in Industrielle Organische Chemie [Industrial Organic Chemistry], 6th edition 2007, Wiley VCH-Verlag, page 275. Finally, the HMD is purified by distillation. Only then can the HMD be converted to polyamines by heating in the presence of transition metal catalysts.

The polycondensation of hexamethylenediamine to give polyhexamethylenepolyamine proceeds in the presence of nickel catalysts as described in WO 92/17437 or palladium catalysts as described in DE 2842264.

A disadvantage is that, for the preparation of polyhexamethylenepolyamines, two reaction steps are required in both processes—a hydrogenation and a polycondensation—with different reaction conditions and catalysts. There is additionally the distillative workup of the HMD.

Further disadvantages are that, in the polycondensation of the HMD in the presence of nickel and palladium as catalysts, colored polyhexamethylenepolyamines as described in WO 92/17437, page 7, table 1 and examples 1 and 2 are obtained, and, in the presence of nickel, polyamines having low average molecular weights as described in WO 92/17437, page 8.

The preparation of polyamines from amino nitriles is also known as a two-stage process. In German published specification DE 2605212, examples 3 and 1 describe the preparation of polypropylenepolyamines from acrylonitrile, ammonia and hydrogen.

First of all, according to example 3, 3-aminopropionitrile prepared from acrylonitrile and ammonia is hydrogenated at 100° C. and a hydrogen pressure of 100 bar in the presence of ammonia over a fixed bed cobalt catalyst to give crude propane-1,3-diamine.

The crude propane-1,3-diamine obtained in example 3 is then, according to example 1, passed continuously over a catalyst composed of reduced cobalt oxide at 160° C. and a hydrogen pressure of 50 bar. This gives 10% by weight of propane-1,3-diamine, 12% dipropylenetriamine, 15% tripropylenetetramine and 70% polypropylenepolyamine of undescribed composition, based on acrylonitrile used.

A disadvantage of this procedure is that the preparation of polypropylenepolyamine from 3-aminoproplonitrile is conducted in two stages at different temperatures and pressures.

DE 3 248 326 A1, page 5 lines 12 to 23, further discloses conducting the hydrogenation of mono-, bis- or tris(2-cyanoethyl)amines I, II and III or mixtures thereof with addition of 5% to 400% by weight of a diamine. The hydrogenation is conducted at 60 to 140° C., preferably 70 to 95° C., and a pressure of 60 to 200 bar, preferably 150 to 200 bar, in the presence of a cobalt catalyst as described at page 5 line 34 to page 6 line 2. The maximum temperature in example 1 is 98° C., and in example 3 96° C.

The hydrogenation as described in DE 3248326 forms the amines IV, V or VI shown in scheme 1, but no polyamines. The addition of diamine is advantageous since any acrylonitrile released during the hydrogenation is scavenged by the diamine. This prevents deactivation of the hydrogenation catalyst by free acrylonitrile and reduction in its service life.

The problem addressed was that of providing a one-stage and hence more economically viable process for preparing polyamines proceeding from dinitriles and/or amino nitriles. In this process, the catalytic hydrogenation of the nitrile group and the polycondensation of the diamines obtained by hydrogenation are to be performable in one stage in the presence of the same catalysts within a narrow temperature range. The polyamines are to be obtained with high average molecular weights with good yield and purity (few by-products, good color number of the polyamines). High average molecular weight means a degree of polymerization Pn of 4 or more, preferably 10 or more, especially preferably 15 or more and most preferably 20 or more. It should be possible to discharge the ammonia formed in the polycondensation from the process.

One-stage preparation is understood to mean that the hydrogenation of the nitriles and the subsequent polycondensation of the diamines are effected in the presence of hydrogen without intermediate workup of the diamines formed at first, and that no change to another catalyst is needed for the hydrogenation and polycondensation. The hydrogenation and polycondensation are conducted here in one to two reactors, preferably in one reactor, and in a temperature range for the two reaction steps of 100° C. to 200° C.

This object is achieved by a process for preparing polyamines, which comprises converting compounds of the formula (I)

NC—Zₘ—(X)ₙ—Y (I)
where
Z corresponds to the CH₂—CH₂—NR— group,
m=0 or 1,
   where in the case that m=0 NC— is bonded directly to X,
   in the case that m=1 and n=1 NR is bonded to X and R=H or CH₂—CH₂—CN, or
   in the case that m=1 and n=0 either R of NR is selected from the group consisting of CH₃, phenyl, CH₂—CH₂—CN and CH₂—CH₂—CH₂—NH₂ or the nitrogen of the NR is part of a heterocycle selected from the group of piperazines, (benz)imidazoles, pyrazoles, triazoles and diazepanes, in which another nitrogen of the heterocycle is bonded to Y,
X is selected from the group of alkyl group-substituted or unsubstituted methylene, ethylene, propylene, butylene, pentylene, hexylene, heptylene, octylene, nonanylene, decanylene, undecanylene, dodecanylene groups, of alkyl group-substituted or unsubstituted 1,2-, 1,3- or 1,4-benzyl groups, 1,4′-bicarbonyl groups, 9,10-anthracenyl groups, alkyl group-substituted or unsubstituted 1,2-, 1,3- or 1,4-cycloalkyl groups, substituted or unsubstituted phenyl-N group, of piperazines, (benz)imidazoles, pyrazoles, triazoles and diazepanes, where the bond to CN or Z and Y or H in each case is via the two nitrogen atoms of the ring, one to two methylene groups that may be substituted by linear or branched C₁- to C₁₂-alkyl, C₆- to C₁₀-aryl, C₇ to C₁₂-aralkyl, C₃- to C₈-cycloalkyl, and at least one methylene group may be replaced by O, NH or NR, where R has the same definition as above and C₁- to C₁₂-alkyl, C₆- to C₁₀-aryl, C₇ to C₂-aralkyl, C₃- to C₈-cycloalkyl groups may in turn be substituted by alkyl groups,
n is 0 or a natural number from 1 to 10,
Y in the case that m=0 and n=1 or higher is CN, CH₂—NH₂ or CH₂—CH₂—CN or
   in the case that m=1 and n=1 or higher is NH₂ or NR—CH₂—CH₂—CN or in the case that m=1 and n=0 is H, CH₂—CH₂—CN, CH₂—CH₂—CH₂—NH₂, alkyl group-substituted or unsubstituted benzyl or cycloalkyl group, or in the case that m=0 and n=0 is CH₂—CN, CH₂—CH₂—CN, CH₂—CH₂—CH₂—CN, CH₂—CH₂—CH₂—CH₂—CN, CH₂—CH₂—CH₂—CH₂—CH₂—CN, CH₂—NH₂, CH₂—CH₂—NH₂, CH₂—CH₂—CH₂—NH₂,
in one stage at temperatures in the range from 100 to 200° C. and pressures in the range from 20 to 200 bar in the presence of hydrogen and a heterogeneous catalyst, the same heterogeneous catalyst if two or more reactors are used, comprising one or more elements of transition group 8 of the Periodic Table.

Preference is given to the process of the invention when the entire process is conducted in a single reactor.

Preference is given to the inventive when each reaction stage of the process is conducted at the same pressure and the same temperature.

Preference is given to the process of the invention when the compounds of the formula (I) are dinitriles of the formula

NC—(—X—)ₙ—CN (Ia)

or

NC—CH₂—CH₂—NR—(—X—)ₙ—NH—CH₂—CH₂—CN (IIa)

where R, n, X are as defined above.

Preference is given to the process of the invention when the compounds of the formula (I) are amino nitriles of the formula

NC—(—X)ₙ—CH₂NH₂, (Ib)

NC—(—X)ₙ (Ib′)

or

NC—CH₂—CH₂—NR—(—X)ₙ—NH₃ (IIb)

where R, n and X are as defined above.

Preference is given to the process of the invention when the elements in the heterogeneous catalysts used are selected from the group of Co, Ni and/or Cu.

Preference is given to the process of the invention when the reduced heterogeneous catalyst used is an unsupported Co catalyst with up to 99% by weight of Co.

Preference is given to the process of the invention when the dinitriles used are selected from the group of malononitrile, succinonitrile, glutaronitrile, adiponitrile, suberonitrile, 2-methylmalononitrile, 2-methylsuccinonitrile, 2-methylglutaronitrile, 1,2-, 1,3- and 1,4-dicyanobenzene, 9,10-anthracenodicarbonitrile and 4,4′-biphenyldicarbonitrile.

Preference is given to the process of the invention when the amino nitriles used are selected from the group of 6-aminocapronitrile, 4′-aminobutyronitrle, 3′-aminopropionitrile and 2′-aminoacetonitrile.

Preference is given to the process of the invention when an excess of hydrogen, based on the amount of hydrogen theoretically needed for hydrogenation of the nitrile groups, of 1 to 5 moles is introduced into the liquid or gas phase of the first reactor (R1), and excess hydrogen together with ammonia formed in the reaction is removed from the last reactor.

Preference is given to the process of the invention wherein the ammonia is separated from the ammonia/hydrogen mixture discharged and discharged from the process, and the hydrogen separated off is wholly or partly recycled into the reactor.

Preference is given to the process of the invention when the ammonia/hydrogen gas mixture is separated by distillation or after cooling by phase separation.

Preference is given to the process of the invention when a portion of the ammonia separated off is recycled into one of the reactors, preferably into the first reactor (R1).

Preference is given to the process of the invention when the process is conducted batchwise or continuously.

Preference is given to the process of the invention when the reactor(s) are tubular reactors or shell and tube reactors.

Preference is given to the process of the invention when the unconverted dinitrile/amino nitrile of the formula (I) from the crude polyamine output and the corresponding diamines and polyamine oligomers thereof are separated off by distillation and recycled into a reactor, preferably the first reactor (R1).

In the formulae (I), (Ia), (Ib), (Ib′), (IIa) and (IIb), the “spacer” X is a group selected from the group of alkyl group-substituted or unsubstituted methylene, ethylene, propylene, butylene, pentylene, hexylene, heptylene, octylene, nonanylene, decanylene, undecanylene, dodecanylene groups, of alkyl group-substituted or unsubstituted 1,2-, 1,3- or 1,4-benzyl groups, 1,4′-bicarbonyl groups, 9,10-anthracenyl groups, alkyl group-substituted or unsubstituted 1,2-, 1,3- or 1,4-cycloalkyl groups, substituted or unsubstituted phenyl-N group, of piperazines, (benz)imidazoles, pyrazoles, triazoles and diazepanes, where the bond to CN or Z and Y or H in each case is via the two nitrogen atoms of the ring, one to two methylene groups that may be substituted by linear or branched C₁- to C₁₂-alkyl, C₆- to C₁₀-aryl, C₇- to C₁₂-aralkyl, C₃- to C₈-cycloalkyl, and at least one methylene group may be replaced by O, NH or NR, where R has the same definition as above and C₁- to C₁₂-alkyl, C₆- to C₁₀-aryl, C₇- to C₁₂-aralkyl, C₃- to Ca-cycloalkyl groups may in turn be substituted by alkyl groups.

Z is CH₂—CH₂—NR.

The index m may be 0 or 1, where in the case that m=0 NC— is bonded directly to X, in the case that m=1 and n=1 NR is bonded to X and R in that case is H or CH₂—CH₂—CN, or in the case that m=1 and n=0 either R of NR is selected from the group consisting of CH₃, phenyl, CH₂—CH₂—CN and CH₂—CH₂—CH₂—NH₂ or the nitrogen of NR is part of a heterocycle in which R comprises at least one further nitrogen atom and this further nitrogen atom is bonded to Y.

Y is dependent on m and n. When m=0, n must be 1 or higher, and in that case Y is selected from the group of CN, CH₂—NH₂ and CH₂—CH₂—NH₂. When m=1 and n=1 or higher, Y is selected from the group of NH₂ and NR—CH₂—CH₂—CN where R is as defined above. When m=1 and n=0, Y is selected from the group of H, CH₂—CH₂—CN and CH₂—CH₂—CH₂—NH₂, alkyl group-substituted or unsubstituted benzyl or cycloalkyl group, or, in the case that m=0 and n=0, CH₂—CN, CH₂—CH₂—CN, CH₂—CH₂—CH₂—CN, CH₂—CH₂—CH₂—CH₂—CN, CH₂—CH₂—CH₂—CH₂—CH₂—CN, CH₂—NH₂, CH₂—CH₂—NH₂, CH₂—CH₂—CH₂—NH₂.

The index n is either 0 or a natural number from 1 to 10.

The compounds of the formula (I) comprise dinitriles of the formula (Ia) and (IIa) and amino nitriles of the formula (Ib), (Ib′) and (IIb).

Dinitriles of the Formula (Ia)

Dinitriles (Ia) required as starting compounds for the process of the invention can be prepared by reacting corresponding alpha,omega- or alpha,beta-dihalogen compounds with alkali metal cyanides, although equimolar amounts of alkali metal halides are formed as by-products. They are also obtainable by dehydration of carboxamides and aldoximes.

The fiber precursor adiponitrile is prepared on the industrial scale by three different processes: by i) reaction of adipic acid with ammonia, ii) electrochemical dimerization of acrylonitrile or iii) by hydrocyanation of butadiene or 3-pentenenitrile with hydrogen cyanide (Ullmann's Encyclopedia of Industrial Chemistry, 6th edition, volume 16, page 434).

Preferred dinitriles (Ia) are compounds in which X denotes 1,2-, 1,3- or 1,4-benzyl groups alkyl-substituted or unsubstituted, 1,2-, 1,3- or 1,4-cycloalkyl groups alkyl-substituted or unsubstituted, methylene groups that may be substituted by C₁- to C₅-alkyl radicals and in which n is 1 to 10. Preferred dinitriles of the formula (Ia) are selected from the group of malononitrile, succinonitrile, glutaronitrile, adiponitrile, suberonitrile, 2-methylmalononitrile, 2-methylsuccinonitrile, 2-methylglutaronitrile, 1,2-, 1,3- and 1,4-dicyanobenzene, 9,10-anthracenodicarbonitrile and 4,4′-biphenyldicarbonitrile. Particular preference is given to adiponitrile, succinonitrile, malononitrile and 2-methylglutaronitrile. Very particularly preferred dinitriles of the formula (Ia) are adiponitrile and malononitrile.

Amino Nitriles of the Formula (Ib) and (Ib′)

Amino nitriles (Ib) required as starting compounds for the process of the invention are obtainable, for example, by partial hydrogenation of dinitriles, for example by partial hydrogenation of adiponitrile to 6-aminocapronitrile in the presence of iron, cobalt or nickel catalysts as described in U.S. Pat. No. 6,297,394. Aminoacetonitriles can be prepared by reacting formaldehyde with hydrogen cyanide and ammonia or alkylamines as described in U.S. Pat. No. 5,008,428. Aminopropionitriles are obtainable by reacting acrylonitrile or methacrylonitrile with ammonia or amines as described in DE 2 605 212 or DE 3 248 326. Preference is given to those amino nitriles that form from the reaction of a primary amine or a secondary amine also comprising at least one further primary amino function with 1 mol of acrylonitrile. Particular preference is given to 3-aminoproplonitrile, N-methylproplonitrile, H₂N(CH₂)₂—NH—(CH₂)₂—CN,

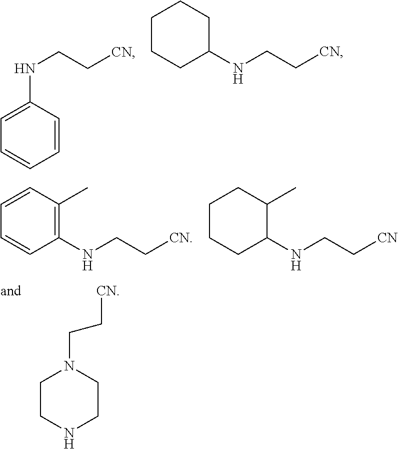

Very particularly preferred dinitriles of the formula (Ib) and (Ib′) are 3-aminopropionitrile, N-methylpropionitrile and 3-piperazin-1-ylpropanenitrile.

Dinitriles (IIa) and Amino Nitriles (IIb)

Dinitriles (IIa) and amino nitriles (IIb) are obtained by reaction of diamines with one mol or two moles of acrylonitrile or methacrylonitrile. This reaction, known as cyanoethylation, can be conducted, for example, in aqueous solution and is described in March's Advanced Organic Chemistry, 6th edition 2007, page 1008, Wiley-Interscience.

Suitable “spacer diamines” X in the formulae (IIa) and (IIb) are fragments of aliphatic diamines, oligomeric polyamines consisting of 2 to 5 amine units and alicyclic and cyclic aromatic diamines, the two amine functions of which have been eliminated by the coupling to Z and Y from the formula (I). X is more preferably a fragment of one of the diamines which follow, minus the two amine functions, selected from the group of ethylenediamine, propylene-1,3-diamine, propylene-1,2-diamine, butylene-1,4-diamine, butylene-1,2-diamine, 1,5-diaminopentane, 1,2-diaminopentane, 1,5-diamino-2-methylpentane, 1,6-diaminohexane, 1,2-diaminohexane, 1,7-diaminoheptane, 1,2-diaminoheptane, 1,8-diaminooctane, 1,2-diaminooctane, 1,9-nonamethylenediamine, 1,10-decamethylenediamine, 1,2-diaminodecane, 1,11-undecamethylenediamine, 1,2-diaminoundecane, 1,12-dodecamethylenediamine, 1,2-diaminododecane, 2,2-dimethylpropane-1,3-diamine, 4,7,10-trioxatridecane-1,13-diamine, 4,9-dioxadodecane-1,12-diamine, N,N-bis(3-aminopropyl)methylamine, N,N-bis(3-aminopropyl)ethylenediamine, 3-(2-aminoethylamino)propylamine, diethylenetriamine (DETA), triethylenetetramine (TETA), tetraethylenepentamine (TEPA), dipropylene-1,3-triamine, tripropylene-1,3-tetramine and tetrapropylene-1,3-pentamine, dipropylene-1,2-triamine, tripropylene-1,2-tetramine and tetrapropylene-1,2-pentamine, dihexamethylenetriamine, trihexamethylenetetramine and tetrahexamethylenepentamine, 3,3′-dimethyl-4,4′-diaminod icyclohexylmethane, 4,4-diaminodicyclohexylmethane, isophoronediamine, 1,3-bis(aminomethyl)cyclohexane, bis(4-aminocyclohexyl)methane, bis(4-amino-3,5-dimethylcyclohexyl)methane or bis(4-amino-3-methylcyclohexyl)methane, 3-(cyclohexylamino)propylamine, piperazine, meta-xylenediamine (MXDA), 4-methylcyclohexane-1,3-diamine, 3-methylcyclohexane-1,2-diamine, 2-methylcyclohexane-1,3-diamine, 4-methylcyclohexane-1,2-diamine, 2-methylcyclohexane-1,4-diamine, 5-methylcyclohexane-1,3-diamine and their cis and trans isomers or isomers of aminobenzylamine (2-aminobenzylamine, 4-aminobenzylamine), 4-(2-aminoethyl)anillne, m-xylylenediamine, o-xylylenediamine, or 2,2-biphenyldiamines, or oxydianilines, for example 4,4-oxydianiline, isomers of diaminofluorene, isomers of diaminophenanthrene and ethylene-4,4-dianiline, Jeffamines from Huntsman, especially Jeffamine D230, Jeffamine D400, Jeffamine D2000, Jeffamine D4000, Jeffamine ED600, Jeffamine ED900, Jeffamine ED2003, Jeffamine EDR148 and Jeffamine EDR176.

X is more preferably a fragment of one of the diamines which follow, minus the two amine functions, selected from the group of propylene-1,3-diamine, 1,6-diaminohexane, 1,8-diaminooctane, 1,10-decamethylenediamine, 1,12-dodecamethylenediamine, 2,2-dimethylpropane-1,3-diamine, N,N-bis(3-aminopropyl)methylamine, N,N-bis(3-aminopropyl)ethylenediamine, dipropylene-1,3-triamine, tripropylene-1,3-tetramine, dihexamethylenetriamine, 4,4-diaminodicyclohexylmethane, isophoronediamine, 1,3-bis(aminomethyl)cyclohexane, piperazine, meta-xylenediamine (MXDA), bis(4-aminocyclohexyl)methane, 3-(cyclohexylamino)propylamine, 4-methylcyclohexane-1,3-diamine, 3-methylcyclohexane-1,2-diamine, 2-methylcyclohexane-1,3-diamine, 4-methylcyclohexane-1,2-diamine, 2-methylcyclohexane-1,4-diamine, 5-methylcyclohexane-1,3-diamine and their cis and trans isomers, m-xylylenediamine, o-xylylenediamine, Jeffamines from Huntsman, especially Jeffamine D230, Jeffamine D400, Jeffamine D2000, Jeffamine D4000, Jeffamine ED600, Jeffamine ED900, Jeffamine ED2003, Jeffamine EDR148 and Jeffamine EDR176.

X is very especially preferably a fragment of one of the diamines which follow, minus the two amine functions, selected from the group of propylene-1,3-diamine, 1,6-diaminohexane, N,N-bis(3-aminopropyl)methylamine, N,N-bis(3-aminopropyl)ethylenediamine, isophoronediamine, piperazine, meta-xylenediamine (MXDA), 4-methylcyclohexane-1,3-diamine, 3-methylcyclohexane-1,2-diamine, 2-methylcyclohexane-1,3-diamine, 4-methylcydohexane-1,2-diamine, 2-methylcyclohexane-1,4-diamine, 5-methylcydohexane-1,3-diamine and their cis and trans isomers.

Catalysts

Catalysts used for the conversion of dinitriles (Ia) and (IIa) and amino nitriles (Ib), (Ib′) and (IIb) to polyamines may especially be catalysts comprising one or more elements of transition group 8 of the Periodic Table (Fe, Co, Ni, Ru, Rh, Pd, Os, Ir, Pt), preferably Co, Ni, Ru, Cu and/or Pd, more preferably Co, Ni and/or Cu, most preferably cobalt. These elements are also referred to hereinafter as catalytically active metals.

The abovementioned catalysts may have been doped with promoters, for example with chromium, iron, cobalt, manganese, molybdenum, titanium, tin, metals of the alkali metal group, metals of the alkaline earth metal group and/or phosphorus.

The catalysts used may preferably be what are called skeletal catalysts (also referred to as Raney-type, hereinafter also: Raney catalysts), which are obtained by leaching out (activating) an alloy composed of catalyst, reactive metal and a further component (preferably Al) with alkali. Preference is given to using Raney nickel or Raney cobalt catalysts.

Catalysts used are further preferably supported catalysts. Preferred support materials are activated carbon, Al₂O₃, TiO₂, ZrO₂ and SiO₂.

In the case of catalysts comprising predominantly cobalt, unsupported cobalt catalysts which comprise up to 99% cobalt but no support and are described in EP-A1 636 409 are very particularly suitable.

Catalysts used in the process according to the invention are most preferably those that are prepared by reduction of what are called catalyst precursors.

The catalyst precursor comprises an active composition comprising one or more catalytically active components, optionally promoters and optionally a support material.

The catalytically active components are oxygen compounds of the abovementioned catalytically active metals, for example the metal oxides or hydroxides thereof, such as CoO, NIO, CuO and/or mixed oxides thereof.

In general, it is only on completion of reduction that catalytic activity occurs in the conversion of the invention.

Particular preference is given to catalyst precursors comprising one or more oxides of the elements Cu, Co and Ni. For example, the oxide mixtures which are disclosed in EP-A-0636409 and which comprise, prior to reduction with hydrogen, 55 to 98% by weight of Co, calculated as CoO, 0.2 to 15% by weight of phosphorus, calculated as H₃PO₄, 0.2 to 15% by weight of manganese, calculated as MnO₂, and 0.2 to 15% by weight of alkali metal, calculated as M₂O (M=alkali metal), or

oxide mixtures which are disclosed in EP-A-0742045 and which comprise, prior to reduction with hydrogen, 55 to 98% by weight of Co, calculated as CoO, 0.2 to 15% by weight of phosphorus, calculated as H₃PO₄, 0.2 to 15% by weight of manganese, calculated as MnO₂, and 0.05 to 5% by weight of alkali metal, calculated as M₂O (M=alkali metal), or

oxide mixtures which are disclosed in EP-A-696572 and which comprise, prior to reduction with hydrogen, 20 to 85% by weight of ZrO₂, 1 to 30% by weight of oxygen compounds of copper, calculated as CuO, 30 to 70% by weight of oxygen compounds of nickel, calculated as NiO, 0.1 to 5% by weight of oxygen compounds of molybdenum, calculated as MoO₃, and 0 to 10% by weight of oxygen compounds of aluminum and/or manganese, calculated as Al₂O₃ and MnO₂ respectively, for example the catalyst disclosed in loc. cit., page 8, with the composition of 31.5% by weight of ZrO₂, 50% by weight of NiO, 17% by weight of CuO and 1.5% by weight of MoO₃, or

oxide mixtures which are disclosed in EP-A-963975 and which comprise, prior to reduction with hydrogen, 22 to 45% by weight of ZrO₂, 1 to 30% by weight of oxygen compounds of copper, calculated as CuO, 15 to 50% by weight of oxygen compounds of nickel, calculated as NiO, where the molar Ni:Cu ratio is greater than 1, 15 to 50% by weight of oxygen compounds of cobalt, calculated as CoO, 0 to 10% by weight of oxygen compounds of aluminum and/or manganese, calculated as Al₂O₃ and MnO₂ respectively, and no oxygen compounds of molybdenum, for example the catalyst A disclosed in loc. cit., page 17, with the composition of 33% by weight of Zr, calculated as ZrO₂, 28% by weight of Ni, calculated as NiO, 11% by weight of Cu, calculated as CuO, and 28% by weight of Co, calculated as CoO.

In a very particularly preferred embodiment, 50 to 100 mol %, more preferably 60 to 99 mol % and most preferably 75 to 98 mol % of the catalytically active metals present in the catalytically active composition are one or more metals selected from the group consisting of Cu, Co and Ni.

The molar ratio of the atoms of the components of the active composition relative to one another can be measured by means of known methods of elemental analysis, for example of atomic absorption spectrometry (AAS), of atomic emission spectrometry (AES), of X-ray fluorescence analysis (XFA) or of ICP-OES (Inductively Coupled Plasma Optical Emission Spectrometry). The molar ratio of the atoms of the components of the active composition relative to one another can also be determined by calculation, for example by determining the starting weights of the compounds used which comprise the components of the active composition and determining the proportions of the atoms in the components of the active composition on the basis of the known stoichiometry of the compounds used, such that it is possible to calculate the atomic ratio from the starting weights and the stoichiometric formula of the compound used. Of course, the stoichiometric formula of the compounds used can also be determined experimentally, for example by one or more of the abovementioned methods.

According to the process conducted (suspension polymerization, fluidized bed process, fixed bed polymerization), the catalysts are used in the form of powder, spall or shaped bodies (preferably extrudates or tablets).

The catalysts or catalyst precursors are preferably used in the form of shaped bodies in the process according to the invention.

Suitable shaped bodies are those having any geometry or shape. Preferred shapes are tablets, rings, cylinders, star extrudates, wagonwheels or spheres. Particular preference is given to tablets, rings, cylinders, spheres or star extrudates. Very particular preference is given to tablets, rings, star extrudates and cylinders.

Impregnation

In a preferred embodiment, the catalysts are used in the process according to the invention in the form of shaped bodies which are prepared by saturation (impregnation) of support materials which have the abovementioned geometry or which are shaped after impregnation to shaped bodies having the abovementioned geometry.

Useful support materials include, for example, carbon such as graphite, carbon black, graphene, carbon nanotubes and/or activated carbon, aluminum oxide (gamma, delta, theta, alpha, kappa, chi or mixtures thereof), silicon dioxide, zirconium dioxide, zeolites, aluminosilicates or mixtures thereof.

The abovementioned support materials can be impregnated by the customary processes as described in A. B. Stiles, Catalyst Manufacture—Laboratory and Commercial Preparations, Marcel Dekker, New York, 1983, for example by application of a metal salt solution in one or more impregnation stages. Useful metal salts generally include water-soluble metal salts such as the nitrates, acetates or chlorides of the corresponding catalytically active components or doping elements, such as cobalt nitrate or cobalt chloride. Thereafter, the impregnated support material is generally dried and optionally calcined.

Calcining is performed generally at temperatures between 300 and 800° C., preferably 350 to 600° C., especially at 450 to 550° C.

The impregnation can also be effected by the “incipient wetness method”, in which the support material is moistened with the impregnation solution up to a maximum of saturation, according to its water absorption capacity. Alternatively, impregnation may take place in supernatant solution.

In the case of multistage impregnation processes, it is appropriate to dry and optionally to calcine between individual impregnation steps. Multistage impregnation should be employed advantageously when the support material is to be contacted with metal salts in a relatively large amount.

For the purpose of applying a plurality of metal components to the support material, impregnation may take place simultaneously with all of the metal salts, or in any desired order of the individual metal salts in succession.

Preference is given to using support materials which already have the above-described preferred geometry of the shaped bodies.

However, it is also possible to use support materials present in the form of powder or spall, and to subject impregnated support materials to shaping.

For example, the impregnated and dried or calcined support material can be conditioned.

The conditioning can be effected, for example, by adjusting the impregnated support material to a particular particle size by grinding.

After grinding, the conditioned, impregnated support material can be mixed with shaping aids such as graphite or stearic acid, and processed further to give shaped bodies.

Standard processes for shaping are described, for example, in Ullmann's Encyclopedia Electronic Release 2000, chapter: “Catalysis and Catalysts”, pages 28-32 and by Ertl et al., Knözinger, Weitkamp, Handbook of Heterogeneous Catalysis, VCH Weinheim, 1997, pages 98 ff.

Standard processes for shaping are, for example, extrusion, tableting, i.e. mechanical pressing, or pelletizing, i.e. compaction by circular and/or rotating movements.

The shaping operation can give shaped bodies with the abovementioned geometry. The conditioning or shaping is generally followed by a heat treatment. The temperatures in the heat treatment typically correspond to the temperatures in the calcination.

Coprecipitation

In a further preferred embodiment, shaped bodies which are produced by coprecipitation of all the components thereof, the catalyst precursors thus precipitated being subjected to a shaping operation, are used in the process according to the Invention.

For this purpose, a soluble compound of the corresponding active component, the doping elements and optionally a soluble compound of a support material in a liquid is admixed while heating and stirring with a precipitant until precipitation is complete.

The liquid used is generally water.

Useful soluble compounds of the active components typically include the corresponding metal salts, such as the nitrates, sulfates, acetates or chlorides, of the aforementioned metals.

The soluble compounds of a support material used are generally water-soluble compounds of Ti, Al, Zr, Si etc., for example the water-soluble nitrates, sulfates, acetates or chlorides of these elements.

The soluble compounds of the doping elements used are generally water-soluble compounds of the doping elements, for example the water-soluble nitrates, sulfates, acetates or chlorides of these elements.

Typically, in the precipitation reactions, the soluble compounds are precipitated as sparingly soluble or insoluble, basic salts by addition of a precipitant.

The precipitants used are preferably alkalis, especially mineral bases, such as alkali metal bases. Examples of precipitants are sodium carbonate, sodium hydroxide, potassium carbonate or potassium hydroxide.

The precipitants used may also be ammonium salts, for example ammonium halides, ammonium carbonate, ammonium hydroxide or ammonium carboxylates.

The precipitation reactions can be conducted, for example, at temperatures of 20 to 100° C., particularly 30 to 90° C., especially at 50 to 70° C.

The precipitates obtained in the precipitation reactions are generally chemically inhomogeneous and generally comprise mixtures of the oxides, oxide hydrates, hydroxides, carbonates and/or hydrogencarbonates of the metals used. With regard to the filterability of the precipitates, it may prove to be favorable for them to be aged—meaning that they are left to themselves for a certain time after precipitation, optionally under hot conditions or with air being passed through.

The precipitates obtained by these precipitation processes are typically processed, by washing, drying, calcining and conditioning them.

After washing, the precipitates are generally dried at 80 to 200° C., preferably 100 to 150° C., and then calcined.

Calcining is performed generally at temperatures between 300 and 800° C., preferably 350 to 600° C., especially at 450 to 550° C.

After the calcination, the pulverulent catalyst precursors obtained by precipitation reactions are typically conditioned.

The conditioning can be effected, for example, by adjusting the precipitation catalyst to a particular particle size by grinding.

After grinding, the catalyst precursor obtained by precipitation reactions can be mixed with shaping assistants such as graphite or stearic acid and processed further to give shaped bodies.

Standard processes for shaping are described, for example, in Ullmann's Encyclopedia Electronic Release 2000, chapter: “Catalysis and Catalysts”, pages 28-32 and by Ertl et al., Ertl, Knözinger, Weitkamp, Handbook of Heterogeneous Catalysis, VCH Weinheim, 1997, pages 98 ff.

Standard processes for shaping are, for example, extrusion, tableting, i.e. mechanical pressing, or pelletizing, i.e. compaction by circular and/or rotating movements.

The shaping operation can give shaped bodies with the abovementioned geometry.

The conditioning or shaping is generally followed by a heat treatment. The temperatures in the heat treatment typically correspond to the temperatures in the calcination.

Precipitative Application

In a further preferred embodiment, the shaped bodies can be produced by precipitative application.

Precipitative application is understood to mean a production method in which a sparingly soluble or insoluble support material is suspended in a liquid and then soluble compounds, such as soluble metal salts, of the corresponding metal oxides are added, and these are then applied by precipitation to the suspended support by addition of a precipitant (for example, described in EP-A2-1 106 600, page 4, and A. B. Stiles, Catalyst Manufacture, Marcel Dekker, Inc., 1983, page 15).

Useful sparingly soluble or insoluble support materials include, for example, carbon compounds such as graphite, carbon black and/or activated carbon, aluminum oxide (gamma, delta, theta, alpha, kappa, chi or mixtures thereof), silicon dioxide, zirconium dioxide, zeolites, aluminosilicates or mixtures thereof.

The support material is generally in the form of powder or spall.

The liquid used, in which the support material is suspended, is typically water.

Useful soluble compounds include the aforementioned soluble compounds of the active components or of the doping elements.

The precipitation reactions can be conducted, for example, at temperatures of 20 to 100° C., particularly 30 to 90° C., especially at 50 to 70° C.

The precipitates obtained in the precipitation reactions are generally chemically inhomogeneous and generally comprise mixtures of the oxides, oxide hydrates, hydroxides, carbonates and/or hydrogencarbonates of the metals used. With regard to the filterability of the precipitates, it may prove to be favorable for them to be aged—meaning that they are left to themselves for a certain time after precipitation, optionally under hot conditions or with air being passed through.

The precipitates obtained by these precipitation processes are typically processed, by washing, drying, calcining and conditioning them.

After washing, the precipitates are generally dried at 80 to 200° C., preferably 100 to 150° C., and then calcined.

Calcining is performed generally at temperatures between 300 and 800° C., preferably 350 to 600° C., especially at 450 to 550° C.

After the calcination, the pulverulent catalyst precursors obtained by precipitation reactions are typically conditioned.

The conditioning can be effected, for example, by adjusting the precipitation catalyst to a particular particle size by grinding.

After grinding, the catalyst precursor obtained by precipitation reactions can be mixed with shaping assistants such as graphite or stearic acid and processed further to give shaped bodies.

Standard processes for shaping are described, for example, in Ullmann's Encyclopedia Electronic Release 2000, chapter: “Catalysis and Catalysts”, pages 28-32 and by Ertl et al., Knözinger, Weitkamp, Handbook of Heterogeneous Catalysis, VCH Weinheim, 1997, pages 98 ff.

Standard processes for shaping are, for example, extrusion, tableting, i.e. mechanical pressing, or pelletizing, i.e. compaction by circular and/or rotating movements.

The shaping operation can give shaped bodies with the abovementioned geometry.

The conditioning or shaping is generally followed by a heat treatment. The temperatures in the heat treatment typically correspond to the temperatures in the calcination.

Reduction

Shaped bodies which have been produced by impregnation or precipitation (precipitative application or coprecipitation) generally comprise the catalytically active components, after calcination, in the form of the oxygen compounds thereof, for example the metal oxides or hydroxides thereof, such as CoO, NiO, CuO and/or the mixed oxides thereof (catalyst precursors).

The catalyst precursors which have been prepared as described above by impregnation or precipitation (precipitative application or coprecipitation) are generally reduced after the calcination or conditioning. The reduction generally converts the catalyst precursor to the catalytically active form thereof.

The reduction of the catalyst precursor can be conducted at elevated temperature in an agitated or unagitated reduction furnace.

The reducing agent used is typically hydrogen or a hydrogen-comprising gas.

The hydrogen is generally used in technical grade purity. The hydrogen can also be used in the form of a hydrogen-comprising gas, i.e. in mixtures with other inert gases, such as nitrogen, helium, neon, argon or carbon dioxide. The hydrogen stream can also be recycled in the reduction as cycle gas, optionally mixed with fresh hydrogen and optionally after removal of water by condensation.

The catalyst precursor is preferably reduced in a reactor in which the shaped bodies are arranged as a fixed bed. Particular preference is given to reducing the catalyst precursor in the same reactor in which the subsequent conversion is effected.

In addition, the catalyst precursor can be reduced in a fluidized bed reactor in the fluidized bed.

The catalyst precursor is generally reduced at reduction temperatures of 50 to 600° C., especially from 100 to 500° C., more preferably from 150 to 450° C. The partial hydrogen pressure is generally from 1 to 300 bar, especially from 1 to 200 bar, more preferably from 1 to 100 bar, the pressure figures here and hereinafter relating to the pressure measured in absolute terms. The duration of the reduction is preferably 1 to 20 hours, and more preferably 5 to 15 hours.

During the reduction, a solvent can be supplied in order to remove water of reaction formed and/or in order, for example, to be able to heat the reactor more quickly and/or to be able to better remove the heat during the reduction. The solvent here may also be supplied in supercritical form.

Suitable solvents used may be the above-described solvents. Preferred solvents are water; ethers such as methyl tert-butyl ether, ethyl tert-butyl ether, dioxane or tetrahydrofuran. Particular preference is given to water or tetrahydrofuran. Suitable solvents likewise include suitable mixtures.

The shaped body thus obtained, after reduction, can be handled under inert conditions. The shaped body can preferably be handled and stored under an inert gas such as nitrogen, or under an inert liquid, for example an alcohol, water or the product of the particular reaction for which the catalyst is used. In that case, it may be necessary to free the catalyst of the inert liquid prior to commencement of the actual reaction.

Storage of the catalyst under inert substances enables uncomplicated and nonhazardous handling and storage of the shaped body.

After reduction, the shaped body can also be contacted with an oxygen-comprising gas stream such as air or a mixture of air with nitrogen.

This gives a passivated shaped body. The passivated shaped body generally has a protective oxide layer. This protective oxide layer simplifies the handling and storage of the catalyst, such that, for example, the installation of the passivated shaped body into the reactor is simplified. A passivated shaped body is preferably reduced as described above by treatment of the passivated catalyst with hydrogen or a hydrogen-comprising gas prior to contacting with the reactants. The reduction conditions generally correspond to the reduction conditions which are employed in the course of reduction of the catalyst precursors. The activation generally removes the protective passivation layer.

Process Parameters

The inventive reaction of dinitriles and amino nitriles of the formula (Ia), (Ib), (Ib′), (IIa) and (IIb) with hydrogen to give polyamines is effected within the temperature range from 100 to 200° C., preferably from 130 to 190° C., more preferably 150 to 180° C.

The reaction is preferably conducted at a pressure at which the reactant/product mixture is in the liquid state. It is effected in the presence of hydrogen at a pressure of 20 to 200 bar, preferably 50 to 190 bar, more preferably 50 to 180 bar.

The catalyst hourly space velocity in continuous mode is generally 0.1 to 2 kg, preferably 0.1 to 1.5 kg, more preferably 0.1 to 1.2 kg, of reactant per L of catalyst and hour, in each case at the prevailing reaction temperature and pressure.

Feeding of the dinitriles, trinitriles and/or amino nitriles of the formula (Ia), (IIa), (Ib), (Ib′), (IIb)

According to the desired composition of the polyamine, the starting compounds (Ia), (IIa), (Ib), (Ib′), (IIb) can be fed to the reactor (R1) individually or as a mixture of two, three or four of the starting compounds.

In batchwise mode, dinitriles, trinitriles and/or amino nitriles of the formula (Ia), (IIa), (Ib), (Ib′), (IIb) are preferably initially charged in the reactor. They can be conveyed into the reactor with suitable conveying apparatus, for example liquid pumps, vacuum conveyors or pneumatic conveyors. Suitable apparatuses for filling a reactor, depending on the state of matter of the substance to be conveyed, are known to those skilled in the art.

The starting compounds are preferably conveyed into the reactor (R1) in the liquid state. For this purpose, it may be necessary to heat them to a temperature above the melting point or solidification point thereof and/or to work under a pressure at which they are in the liquid state. In addition, it may be preferable to dissolve the starting compounds in a solvent.

Suitable liquids are, for example, liquids which are very substantially inert under the conditions of the conversion.

Preferred liquids are C4 to C12 dialkyl ethers such as diethyl ether, diisopropyl ether, dibutyl ether, tert-butyl methyl ether or cyclic C4 to C12 ethers such as tetrahydrofuran, 2- and 3-methyltetrahydrofuran or dioxane, dimethoxyethane, diethylene glycol dimethyl ether, or hydrocarbons such as pentane, hexane, heptane, 2,2,4-trimethylpentane, octane, cyclohexane, methylcyclohexane, xylene, toluene or ethylbenzene, or amides such as formamide, dimethylformamide or N-methylpyrrolidone.

Preference is given to performing the inventive conversion in substance without solvent.

In the case of a continuously operated reactor (R1), the reactants are preferably pumped into the reactor in the liquid state. The flow of the feedstocks into the reactor may be from the top downward (trickle mode) or from the bottom upward (liquid phase mode).

Feeding of Hydrogen

The reactor (R1) in which the dinitriles, trinitriles and/or amino nitriles of the formula (Ia), (IIa), (Ib), (Ib′), (IIb) are converted to polyamines is fed with hydrogen. In the process of the invention, hydrogen has a number of advantageous properties. First of all, hydrogen serves to hydrogenate the nitrile groups to amino groups. In addition, the hydrogen keeps the hydrogenation catalyst active.

In a preferred embodiment of the batchwise and continuous reaction of the invention, the conversion of the dinitriles, trinitriles and/or amino nitriles of the formula (Ia), (IIa), (Ib), (Ib′), (IIb) used, the selectivity and the average molar mass of the polyamines obtained can be distinctly increased by passing excess hydrogen through the first reactor (R1) at constant pressure. Excess hydrogen is understood here to mean that the amount of hydrogen fed in is much greater than the amount needed for the nitrile group hydrogenation (see examples 1a to 1c). The excess molar amount of hydrogen, based on the amount of hydrogen theoretically needed to hydrogenate the nitrile groups, is 0.1 to 20 moles, preferably 1 to 10 moles, more preferably 1 to 5 moles. The flowing hydrogen removes the ammonia released in the polycondensation from the reactor.

The hydrogen used is generally used in technical grade purity. The hydrogen can also be used in the form of a hydrogen-comprising gas, i.e. with additions of other inert gases, such as nitrogen, helium, neon, argon or carbon dioxide. Hydrogen-comprising gases used may, for example, be reformer offgases, refinery gases etc., provided that these gases do not comprise any catalyst poisons for the catalysts used, for example carbon monoxide. However, preference is given to using pure hydrogen or essentially pure hydrogen in the process, for example hydrogen having a content of more than 99% by weight, preferably more than 99.9% by weight, of hydrogen, more preferably more than 99.99% by weight, especially more than 99.999% by weight.

By varying the amount of hydrogen that flows through the reactor, it is possible to remove the entire amount of ammonia released or only a portion of this amount of ammonia from the reactor.

Rather than hydrogen, it is also possible to use mixtures of hydrogen and one or more Inert gases. Hydrogen or mixtures of hydrogen and an inert gas are introduced into the liquid phase or the gas phase, preferably into the liquid phase.

The ammonia is separated by cooling or distillation from the gas mixtures obtained in the case of passage of hydrogen or mixtures of hydrogen and inert gas, and very predominantly discharged from the process. Since nitrile hydrogenations, however, are advantageously conducted in the presence of basic compounds such as ammonia or alkali metal hydroxides (avoidance of the formation of tertiary amines), it is optionally also possible to recycle a portion of the ammonia into the reactor (R1). Unconsumed hydrogen or mixtures of hydrogen and inert gas are recycled wholly or partly into the first or second reactor, preferably into the first reactor (R1).

Inert gases refer hereinafter to gases which are predominantly inert under the present reaction conditions and essentially do not react with the compounds present in the reaction mixture. The inert gases used are preferably nitrogen or the noble gases helium, neon, argon or xenon. Very particular preference is given to using nitrogen. The inert gases used may also be mixtures of the aforementioned gases.

Reactor (R1)

The preparation of the polyamines in the presence of the catalysts mentioned is preferably conducted in the customary reaction vessels suitable for catalysis, in fixed bed or suspension mode, continuously, batchwise or semicontinuously, in one reactor (R1) or two reactors (R1) and (R2) connected in series.

Fixed Bed

In a particularly preferred embodiment, the reaction of the invention is conducted in a reactor in which the catalyst is arranged as a fixed bed. Suitable fixed bed reactors are described, for example, in the article “Catalytic Fixed-Bed Reactors” in Ullmann's Encyclopedia of Industrial Chemistry, Published Online: 15 Jun. 2000, DOI: 10.1002/14356007.b04-199.

Preference is given to performing the process in a shaft reactor, a shell and tube reactor or, more preferably, in a tubular reactor.

Tubular reactors may be used in each case as single reactors, as a series of single reactors and/or in the form of two or more parallel reactors. Preference is given to working with two reactors, particular preference to working with one reactor. If Just one reactor is used, it may be advantageous to divide the reactor into two temperature zones. In this case, the nitrile hydrogenation takes place in the region of the lower temperature, the polycondensation in the region of the higher temperature.

Suspension

In a preferred embodiment, the catalyst is suspended in the reaction mixture to be polymerized. Polymerization in suspension mode can preferably be conducted in a stirred reactor, jet loop reactor, jet nozzle reactor, bubble column reactor, or in a cascade of such identical or different reactors.

Particular preference is given to performing the polycondensation in suspension mode in a stirred reactor.

By contrast with polycondensation of diamines, the reactions proceeding from dinitriles, trinitriles and/or amino nitriles of the formula (Ia), (IIa), (Ib), (Ib′), (IIb), the nitrile groups of which are first hydrogenated, are highly exothermic reactions. Therefore, it is necessary to provide cooling apparatuses in the reactors and/or in product circulation streams.

Workup of the Polyamines

Workup—Column K1

In a very particularly preferred embodiment, the reaction output is decompressed into a distillation column.

The column is generally operated such that ammonia and gas supplied are drawn off at the top of the column and the rest of the liquid phase (monomer, oligomers and polymers) is drawn off at the bottom of the column (variant 1).

Alternatively, column K1 can be operated in such a way that ammonia and the gas supplied are drawn off at the top, monomeric and oligomeric dinitrile/trinitrile/amino nitrile of the formula (I) and the corresponding diamines thereof are drawn off from a side draw in the middle region of the column, and higher molecular weight polyamine is drawn off at the bottom of the column (variant 2).

The exact operating conditions of the distillation column can, in accordance with the separation performance of the column used, be determined in a routine manner by the person skilled in the art by customary calculation methods using the known vapor pressures and evaporation equilibria of the components introduced into the distillation column.

### VARIANT 1

The reactor output is preferably expanded into the middle region of a distillation column K1.

The distillation column K1 is more preferably executed in a tray column. In a tray column, there are intermediate trays on which the mass transfer takes place within the column. Examples of different tray types are sieve trays, tunnel-cap trays, dual-flow trays, bubble-cap trays or valve trays.

In a further preferred embodiment, the distillative internals may take the form of an ordered packing, for example of a sheet metal packing, such as Mellapak 250 Y or Montz Pak, B1-250 type, or of a structured ceramic packing or of an unordered packing, for example composed of Pall rings, IMTP rings (from Koch-Glitsch), Raschig Superrings, etc.

At the top of column K1, a gaseous stream composed of the gas supplied and ammonia is generally obtained.

In a particularly preferred embodiment, ammonia is separated from the gas stream obtained at the top. The separation of ammonia from the gas stream discharged can preferably be effected by cooling the gas stream by means of a cooling apparatus to a temperature at which ammonia is converted to the liquid state, and the gas supplied remains in the gas phase. The cooling apparatus is preferably a condenser.

The condenser of the distillation column K1 is generally operated at a temperature at which the ammonia is very substantially condensed at the corresponding top pressure.

The condensed ammonia is preferably discharged from the process.

The uncondensed gas, which consists essentially of inert gas and/or hydrogen, is preferably recycled into the process. The recycled gas is preferably essentially free of ammonia.

Column K1 generally does not require any additional evaporator at the bottom of the column, since the difference between the boiling points of ammonia and monomeric dinitrile/trinitrile/amino nitrile of the formula (I) and the corresponding diamines thereof is generally sufficiently high that good separation of ammonia and monomeric dinitrile/trinitrile/amino nitrile of the formula (I) and the corresponding diamines thereof is possible without additional heating at the bottom.

However, it is also possible to heat the bottom of the column, for example with a reboiler.

In that case, the temperature at the bottom of the column should be adjusted such that ammonia is very substantially evaporated at the top pressure that exists in the column, while monomeric diamine remains in the liquid phase.

The bottoms discharge from column K1 comprises essentially dinitrile/trinitrile/amino nitrile of the formula (I) and the corresponding diamines thereof, polyamines and optionally solvent.

A portion of the bottoms output can be
a) recycled to the reactor, or
b) introduced into a further column K2 in which monomeric dinitrile/trinitrile/amino nitrile of the formula (I) and the corresponding diamines thereof and low-boiling oligomer are separated from higher-boiling polyamine, or
c) withdrawn from the reactor as reaction product.
d) A portion of the bottoms output from column K1 can be recycled into the reactor, where further condensation takes place. Thus, polymers having a particularly high molecular weight can be achieved.

It is preferable that the bottoms output recycled comprises essentially no ammonia. This is generally already achieved after the flash evaporation (distillation). Should the ammonia contents nevertheless be higher, the ammonia content can be reduced, for example by distillation or degassing (stripping).
b) The bottoms output from column K1 can be introduced into a further distillation column K2, which is operated in such a way that monomeric dinitrile/trinitrile/amino nitrile of the formula (I) and the corresponding diamines thereof and low-boiling oligopolyamine are obtained at the top of the column, and polyamine is drawn off at the bottom of the column. Column K2 is described in detail below.
c) A portion of the bottom product from column K1 can be discharged from the process as reaction product.

### VARIANT 2

Column K1 can also be operated in such a way that ammonia and the gas supplied are obtained at the top of the column, a fraction comprising monomeric dinitrile/trinitrile/amino nitrile of the formula (I) and the corresponding diamines thereof and low-boiling oligomers is withdrawn as a side draw product in the middle region, and polyamine is obtained at the bottom of column K1.

The reactor output is, as in the above-described variant 1, preferably decompressed Into the middle region of a distillation column K1 as described above.

At the top of column K1, a gaseous stream composed of the gas supplied and ammonia is generally obtained.

In a particularly preferred embodiment, ammonia is separated from the gas stream obtained at the top. The separation of ammonia from the gas stream discharged can preferably be effected by cooling the gas stream by means of a cooling apparatus to a temperature at which ammonia is converted to the liquid state, and the gas supplied remains in the gas phase. The cooling apparatus is preferably a condenser.

The condenser of the distillation column K1 is generally operated at a temperature at which ammonia is very substantially condensed at the corresponding top pressure.

The condensed ammonia is preferably discharged from the process.

The uncondensed gas, which consists essentially of inert gas and/or hydrogen, is preferably recycled into the process.

Column K1 generally does not require any additional evaporator at the bottom of the column, since the difference between the boiling points of ammonia and monomeric dinitrile/trinitrile/amino nitrile of the formula (I) and the corresponding diamines thereof is generally sufficiently high that good separation of ammonia and monomeric dinitrile/trinitrile/amino nitrile of the formula (I) and the corresponding diamines thereof is possible without additional heating at the bottom.

However, it is also possible to heat the bottom of the column, for example with a reboller.

The temperature at the bottom of the column should be adjusted such that ammonia is very substantially evaporated at the top pressure that exists in the column, while monomeric dinitrile/trinitrile/amino nitrile of the formula (I) and the corresponding diamines thereof remain in the liquid phase.

The side draw product drawn off from column K1 is preferably a fraction comprising essentially oligomers of the dinitrile/trinitrile/amino nitrile of the formula (I) and the corresponding diamines thereof.

The side draw product can be
a) discharged from the process, or
b) recycled into the process (preferred variant).

When the side draw product is recycled into the process, it is preferable that the side draw product comprises essentially no ammonia. This is generally already achieved after the flash evaporation (distillation). Should the ammonia contents nevertheless be higher, the ammonia content can be reduced, for example by distillation or degassing (stripping).

The bottoms output from column K1 comprises essentially dinitrile/trinitrile/amino nitrile of the formula (I) and the corresponding diamines thereof, oligomers of the dinitrile/trinitrile/amino nitrile of the formula (I) and the corresponding diamines thereof, polyamine and optionally solvent.

A portion of the bottoms output can, as described in variant 1, be
a) recycled to the reactor, or
b) introduced into a further column K2 in which monomeric dinitrile/trinitrile/amino nitrile of the formula (I) and the corresponding diamines thereof and low-boiling oligomer are separated from higher-boiling polyamine, or
c) withdrawn from the reactor as reaction product.

Workup—Column K2

The bottoms output from column K1 can be introduced into a further column K2, which is operated in such a way that monomeric dinitrile/trinitrile/amino nitrile of the formula (I) and the corresponding diamines thereof and low-boiling oligomers are obtained at the top of the column. Column K2 can alternatively be operated in such a way that predominantly monomeric dinitrile/trinitrile/amino nitrile of the formula (I) and the corresponding diamines thereof can be drawn off at the top, predominantly oligomeric dinitrile/trinitrile/amino nitrile of the formula (I) and the corresponding diamines thereof at a side draw, and polymeric dinitrile/trinitrile/amino nitrile of the formula (I) and the corresponding diamines thereof at the bottom.

The bottoms output from column K1 is preferably supplied to the middle region of a distillation column K2.

Preferably, the distillation column K2 has internals for increasing the separation performance. The distillative internals may be present, for example, as an ordered packing, for example as a sheet metal packing such as Mellapak 250 Y or Montz Pak, B1-250 type. It is also possible for a packing with lower or elevated specific surface area to be present, or it is possible to use a fabric packing or a packing with another geometry such as Mellapak 252 Y. What are advantageous about the use of these distillative internals are the low pressure drop and the low specific liquid holdup compared to valve trays, for example. The internals may be disposed in one or more beds.

The bottom of column K2 is preferably equipped with a reboller.

The temperature in the bottom of the column should be adjusted such that ammonia, monomeric dinitrile/trinitrile/amino nitrile of the formula (I) and the corresponding diamines thereof are very substantially evaporated and a portion of the oligomers is evaporated at the top pressure that exists in the column, while polymeric polyamine remains in the liquid phase.

At the top of column K2, in general, a gaseous stream consisting essentially of monomeric dinitrile/trinitrile/amino nitrile of the formula (I) and the corresponding diamines thereof is drawn off.

Preferably, the gas stream obtained at the top is fed to a condenser connected to the distillation column K2.

The condenser of the distillation column K2 is generally operated at a temperature at which the dinitrile/trinitrile/amino nitrile of the formula (I) and the corresponding diamines thereof are very substantially condensed at the corresponding top pressure.

The condensate from column K2, which consists essentially of monomeric dinitrile/trinitrile/amino nitrile of the formula (I) and the corresponding diamines thereof, can be discharged or recycled into the process.

The recycled dinitrile/trinitrile/amino nitrile of the formula (I) and the corresponding diamines thereof preferably comprise essentially no ammonia.

This has the advantage that polyamines having high molecular weight and low degrees of branching can be obtained. In addition, the reaction time until attainment of a certain degree of conversion can be reduced (increased reaction rate). Should the ammonia content be relatively high, the diamine can be subjected to a further distillation or degassing operation, for example to a stripping operation.

A portion of the dinitrile/trinitrile/amino nitrile of the formula (I) and the corresponding diamines thereof obtained as condensate can be recycled into the column as reflux.

A portion of the bottoms output can be recycled to the reactor, or withdrawn from the reactor as reaction product. Preferably, the bottom product from column K2 is discharged as reaction product.

In column K2 it is also possible to withdraw a side draw product comprising a fraction composed of low-boiling oligomers. These oligomers can be discharged, or recycled Into the reactor together with the dinitrile/trinitrile/amino nitrile of the formula (I) and the corresponding diamines thereof that have been discharged at the top.

Preferred Process Variants

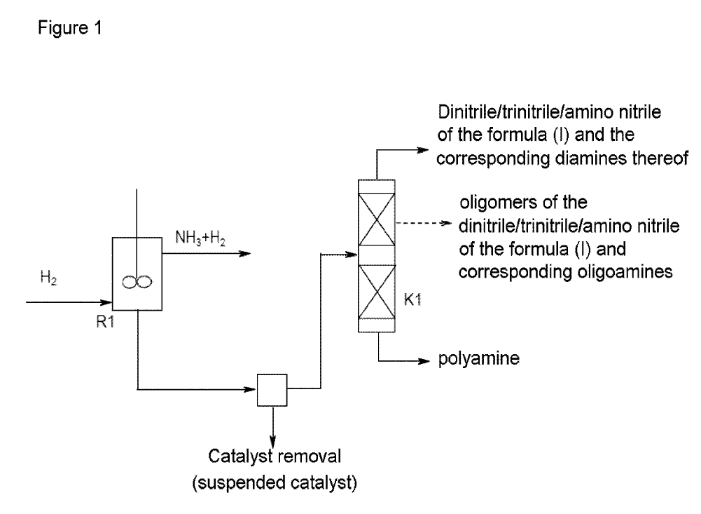

FIGS. 1 to 6 describe particular embodiments of the process according to the invention.

### VARIANT D-1

FIG. 1 shows a batchwise process in which monomer is initially charged in a stirred tank reactor R 1 comprising the catalyst in suspended or fixed form, for example in a metal mesh. Then inert gas and/or hydrogen is passed in continuously. The introduction is preferably effected through a gas inlet tube, a gas distributor ring or a nozzle, which is preferably arranged below a stirrer. The gas stream introduced is broken up into small gas bubbles by the energy input of the stirrer and distributed homogeneously in the reactor. A mixture of ammonia formed and inert gas and/or hydrogen is discharged continuously from the reactor through an outlet orifice in the upper region of the reactor.

If the batchwise hydrogenation and polycondensation is conducted not in the presence of a fixed catalyst but of a suspended catalyst, the suspension catalyst is first removed, for example by filtration or centrifugation, when the product is discharged in the course of workup of the product of value.

The reaction output obtained in the batchwise polycondensation can be passed into a distillation column K1 in which a stream of dinitrile/trinitrile/amino nitrile of the formula (I) and the corresponding diamines thereof and oligomers of the dinitrile/trinitrile/amino nitrile of the formula (I) and the corresponding diamines thereof is removed at the top. Polyamine is obtained at the bottom of the column.

The reaction output obtained in the batchwise polycondensation can alternatively be passed into a distillation column K1 in which a stream of dinitrile/trinitrile/amino nitrile of the formula (I) and the corresponding diamines thereof is removed at the top, and a fraction consisting essentially of oligomers of the dinitrile/trinitrile/amino nitrile of the formula (I) and the corresponding diamines thereof as a side draw. At the bottom of the column, polyamine is drawn off.

### VARIANT D-2

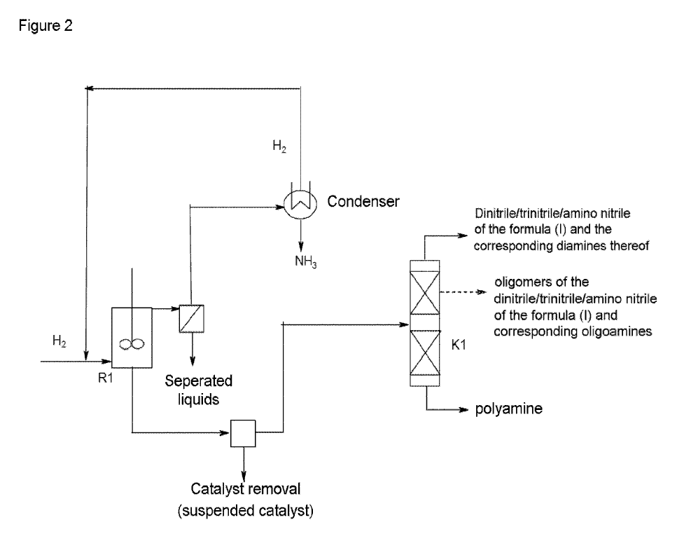

FIG. 2 shows a variant of the process in which the discharged gas stream is decompressed after discharge. For removal of entrained liquid, the gas stream drawn off is introduced into a liquid separator. The liquid separated out in the liquid separator is discharged from the process. Downstream of the liquid separator, the mixture of ammonia and inert gas and/or hydrogen discharged from the reactor is preferably cooled, which liquefles the ammonia, allowing it to be separated from the inert gas and/or hydrogen. The inert gas and/or hydrogen can be compressed again, if necessary admixed with fresh inert gas and/or hydrogen, and recycled into the polymerization stage.

### VARIANT D-3

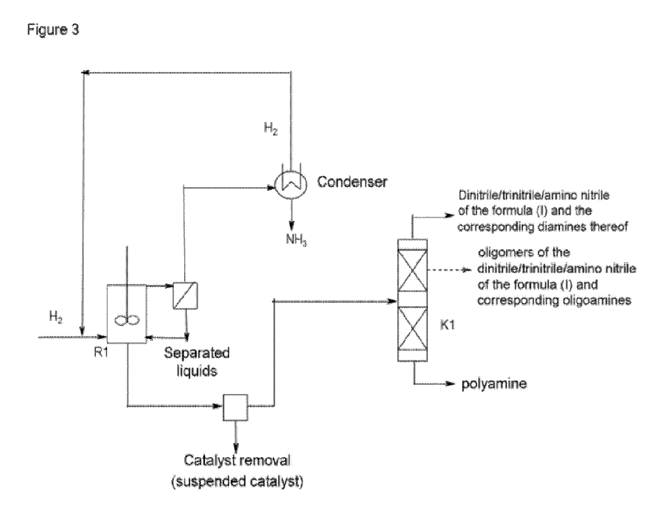

FIG. 3 shows a further variant in which the liquid separated out in the liquid separator, consisting essentially of dinitrile/trinitrile/amino nitrile of the formula (I) and the corresponding diamines thereof, oligomers of dinitrile/trinitrile/amino nitrile of the formula (I) and the corresponding diamines thereof and optionally solvent, is recycled into the process. If the mixture of dinitrile/trinitrile/amino nitrile of the formula (I) and the corresponding diamines thereof and/or oligomers of the dinitriles/trinitriles/amino nitriles of the formula (I) and the corresponding diamines thereof should comprise by-products, these can be separated, for example by distillation, from the dinitriles/trinitriles/amino nitriles of the formula (I) and the corresponding diamines thereof and oligomers thereof prior to their recycling

### VARIANT K-1

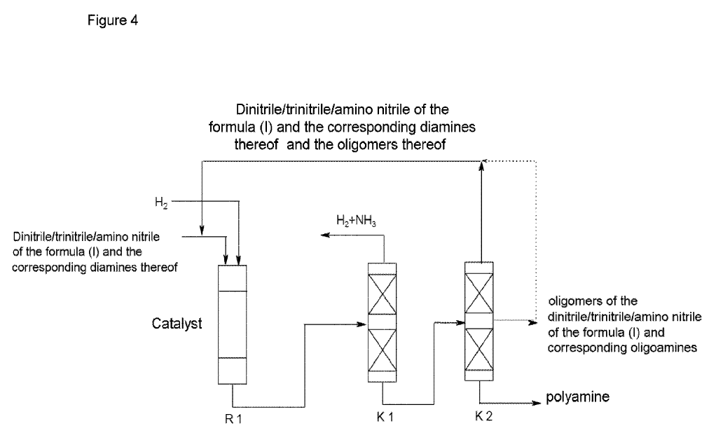

FIG. 4 shows a continuous process for preparing polyamines. Dinitrile/trinitrile/amino nitrile of the formula (I) and the corresponding diamines thereof are passed together with inert gas and/or hydrogen over a catalyst arranged in fixed bed form in an inertized pressure reactor R1.

The reaction output is passed to column K1. A mixture of ammonia and hydrogen is the overhead product from column K1, and this is discharged from the process. The bottom product of column K1 is conducted to a column K2. Unconverted dinitrile/trinitrile/amino nitrile of the formula (I) and the corresponding diamines thereof is the overhead product from column K2, and is recycled into the reactor R1. Oligomers are optionally drawn off from a side draw from column K2, and these are discharged and/or recycled into the reactor R1. The bottom product of column K2 comprises polyamine.

### VARIANT K-2

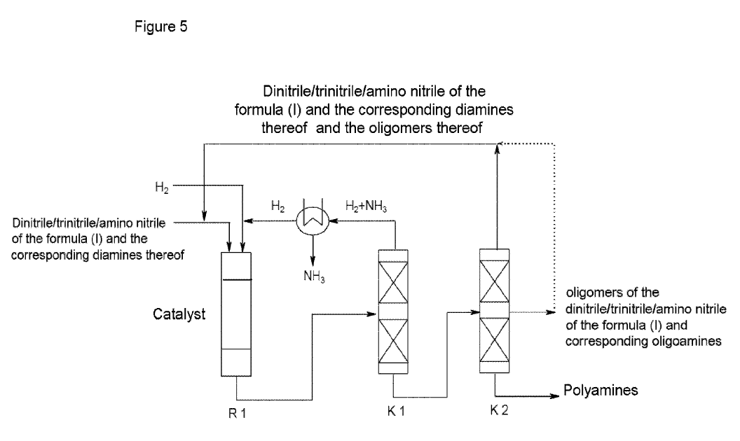

FIG. 5 shows a continuous process for preparing polyamines. Dinitrile/trinitrile/amino nitrile of the formula (I) and the corresponding diamines thereof are passed together with inert gas and/or hydrogen over a catalyst arranged in fixed bed form in an inertized pressure reactor R1.

The reaction output is passed to column K1. The overhead product of column K1 is a mixture of ammonia and hydrogen, out of which the ammonia is condensed. Inert gas and/or hydrogen can be recycled into the reactor R1.

The bottom product of column K1 is conducted to a column K2. Unconverted dinitrile/trinitrile/amino nitrile of the formula (I) and the corresponding diamines thereof and low-boiling oligomer are the overhead product from column K2, and are recycled into the reactor R1. Oligomers are optionally drawn off from a side draw of column K2, and these are discharged and/or recycled into the reactor R1. The bottom product of column K2 comprises polyamine.

### VARIANT K-3

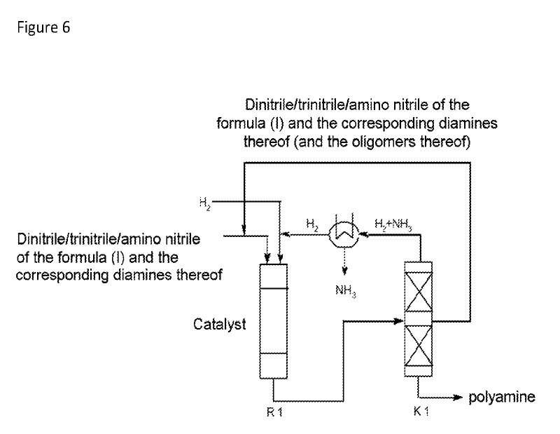

FIG. 6 shows a variant of the continuous process.

Dinitrile/trinitrile/amino nitrile of the formula (I) and the corresponding diamines thereof are passed together with inert gas and/or hydrogen over a catalyst arranged in fixed bed form in an inertized pressure reactor R1. Under the reaction conditions, a reaction output is formed, which is passed to a column K1. Column K1 is operated in such a way that the top product obtained is a mixture of ammonia and inert gas and/or hydrogen mixture, a mixture of dinitrile/trinitrile/amino nitrile of the formula (I) and the corresponding diamines thereof and oligomers of the dinitrile/trinitrile/amino nitrile of the formula (I) and the corresponding diamines thereof is withdrawn from a side draw, and polyamine is withdrawn as bottom product. Column K2 in FIG. 4 or 5 is dispensed with.

Any unconverted dinitrile/trinitrile/amino nitrile of the formula (I) and the corresponding diamines thereof and oligomeric polyamines can be separated off by distillation and recycled into the polyamine synthesis stage.

### 6. WORKING EXAMPLES

### 6.1. One-Stage Preparation of Polyhexamethylenepolyamine from Adiponitrile (Batchwise Mode)

The experiments were conducted in a 300 mL steel pressure vessel stirred with a paddle stirrer. Hydrogen was fed in via an inlet tube. In the upper part of the pressure vessel, it was optionally possible to lead off offgas, which was conducted without cooling into the middle of a vertical steel tube (internal diameter 1.4 cm, height 16 cm). Liquid condensate obtained here was recycled into the lower part of the pressure vessel, and offgas was led off from the apparatus via the steel tube.

The catalyst used was a cobalt catalyst having a strand diameter of 4 mm. Preparation thereof is described in EP-A-0636409. The shaped catalyst bodies were reduced at 280° C. and standard pressure by means of a continuous hydrogen stream of 50 L (STP) per hour for 12 hours. As feedstock, 50 g of adiponitrile (ADN) were initially charged in the pressure vessel under nitrogen. 50 g of the activated catalyst were fixed in a “metal cage”, through which the stirred reaction mixture flowed. The polycondensation was effected at 160° C. and total pressure 60 bar for 4 hours. After the reaction time, the autoclave was cooled to room temperature and decompressed. The reaction mixture was removed from the autoclave. The reaction outputs were analyzed by gas chromatography (% by weight) and by gel permeation chromatography (absolute calibration by measurement of defined polyamine standards).

### Example 1a

The experiment was conducted as described above. In the pressure vessel, a total pressure of 60 bar was maintained by injection of hydrogen over the entire experimental period. No offgas was led out of the pressure vessel. Analysis of the reaction output (table 1) shows that just 11% polyamines (high boilers) had formed.

### Example 1b

The experiment was conducted as described above. During the reaction time of four hours, however, 2.4 moles of hydrogen per mole of ADN per hour were passed continuously through the pressure vessel at a pressure of 60 bar and disposed of. Liquid condensate was recycled into the pressure vessel. The structures of the hexamethyleneimine (HMI), aminohexyl-HMI, HMI-HMD-HMI, bis-HMD and HMI-(HMD)₂ by-products are shown in FIG. 1.

TABLE 1

| | | | | | | HMI- | | | | | |
|---|---|---|---|---|---|---|---|---|---|---|---|
| | T | | | Amino- | Bis- | HMD- | HMI- | | High | | |
| Ex. | [° C.] | HMI | HMD | hexyl-HMI | HMD | HMI | (HMD)2 | Others | boilers | M_{w} | PDI |
| 1a | 160 | 13 | 47 | 1 | 23 | — | — | 5 | 11 | — | — |
| 1b | 160 | 9 | 2 | 6 | 9 | 1 | 4 | 7 | 62 | 750 | 1.6 |

FIG. 1

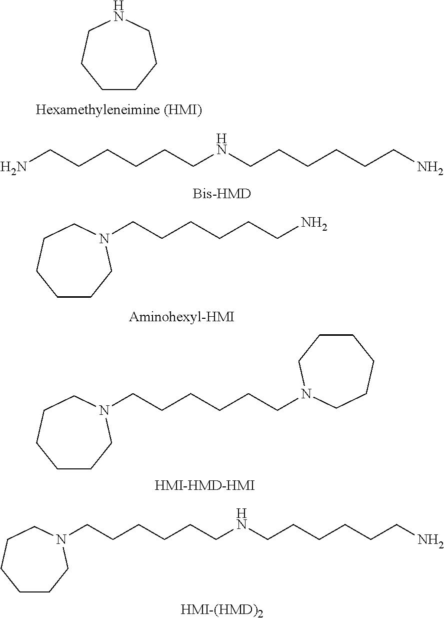

Analysis of the reaction output (table 1) demonstrates that the passage of hydrogen with removal of ammonia formed considerably increases the conversion and leads to average molar masses of 750 g/mol.

### 6.2. One-Stage Preparation of Polyhexamethylenepolyamine from Adiponitrile (Continuous Mode)

### Example 1c

Adiponitrile was passed continuously from the bottom upward through a catalyst present in a tubular reactor. The catalyst used was 378 g of activated cobalt catalyst having a strand diameter of 4 mm, the preparation of which is described in EP-A-0636409 as catalyst A on page 3 lines 35-44. The pressure was 50 bar, the temperature 170° C. The catalyst hourly space velocity was 0.1 kg/Lh of adiponitrile. 5 moles of hydrogen per mole of adiponitrile were passed through the reactor (offgas mode). The crude output was a white, waxy solid. The composition of the crude discharge is summarized in Table 2. The molar mass was determined after distillative removal of HMI, HMD, aminohexyl-HMI and bis-HMD.

Table 2

TABLE 2

| | | | Amino- | | HMI- | | | | | |
|---|---|---|---|---|---|---|---|---|---|---|
| | | | hexyl- | Bis- | HMD- | HMI- | | High | | |
| T [° C] | HMI | HMD | HMI | HMD | HMI | (HMD)2 | Others | boilers | M_{w} | PDI |
| 170 | 1 | 0 | 2 | 0 | 9 | 1 | 14 | 73 | 1996 | 1.7 |

### 6.3. One-Stage Preparation of Polyoctamethylenepolyamine from Suberonitrile (Batchwise Mode)

### Example 2

The experiment was conducted analogously to example 1b. The starting material used was suberonitrile. Over the four hours of reaction time, 1.8 moles of hydrogen per mole of adiponitrile per hour were passed continuously through the pressure vessel and disposed of. Liquid condensate was recycled into the pressure vessel.

The reaction outputs were analyzed by gas chromatography (% by weight) and by gel permeation chromatography (absolute calibration by measurement of defined polyamine standards). The analysis results are summarized in table 3.

TABLE 3

| | | | | | High | | |
|---|---|---|---|---|---|---|---|
| T [° C.] | 1,8-Diaminooctane | Suberonitrile | Dimer | Others | boilers | Mw | PDI |
| 160 | 17 | 0 | 20 | 3 | 60 | 570 | 3.2 |

### 6.4. One-Stage Preparation of Polyhexamethylenepolyamine from 6-Aminocapronitrile (6-ACN) (Continuous Mode)

### Example 3

The experiment was conducted analogously to example 1c. 6-Amlnocapronitrile was passed continuously from the bottom upward through a catalyst present in a tubular reactor. The catalyst used was 378 g of activated cobalt catalyst having a strand diameter of 4 mm, the preparation of which is described in EP-A-0636409 as catalyst A on page 3 lines 35-44. The pressure was 50 bar, the temperature 170° C. The catalyst hourly space velocity was 0.1 kg/Lh of 6-aminocapronitrile. 2 moles of hydrogen per mole of 6-aminocapronitrile were passed through the reactor (offgas mode). The crude output was a white, waxy solid. The composition of the crude output is summarized in table 4. The molar mass was determined after distillative removal of HMI, HMD, aminohexyl-HMI and bis-HMD.

TABLE 4

| | | | | | HMI- | | | | | |
|---|---|---|---|---|---|---|---|---|---|---|
| T | | | Amino- | Bis- | HMD- | HMI- | | High | | |
| [° C.] | HMI | HMD | hexyl-HMI | HMD | HMI | (HMD)2 | Others | boilers | M_{w} | PDI |
| 170 | 3 | 1 | 4 | 1 | 5 | 3 | 8 | 75 | 1634 | 1.8 |

Analysis of the reaction output showed that, with complete 6-ACN conversion and virtually complete HMD conversion, polyhexamethylenepolyamine molar masses of around 1634 g/mol were attained.

### 6.5. One-Stage Preparation of Polyamine from Dicyanoethylated Isophoronediamine (IPDA) (Continuous Mode)

### Example 4

The experiment was conducted analogously to the one-stage preparation of polyhexamethylenepolyamine in continuous mode (analogously to experiment 1c). For this purpose, dicyanoethylated IPDA was passed continuously from the bottom upward through a catalyst. The catalyst used was 378 g of activated cobalt catalyst having a strand diameter of 4 mm, the preparation of which is described in EP-A-0636409 as catalyst A on page 3 lines 35-44. The pressure was 50 bar, the temperature 160° C. The reaction was conducted in the presence of THF as solvent. The reactant was dissolved in 50% by weight of THF. The catalyst hourly space velocity was 0.1 kg/Lh of reactant dissolved in THF. 12 moles of hydrogen per mole of cyanoethylated IPDA were passed through the reactor (offgas mode). The crude output was viscous and clear. The composition of the crude output is summarized in table 5. The molar mass was determined after distillative removal of THF, 3,3,5-trimethyl-7-azabicyclo[3.2.1]octane (IPDA-ring) and IPDA.

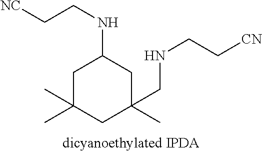 [SMILES: CC1(C)CC(NCCC#N)CC(C)(CNCCC#N)C1]

TABLE 5

| | | | | | High | | |
|---|---|---|---|---|---|---|---|
| T [° C.] | THF | IPDA-Ring | IPDA | Others | boilers | Mw | PDI |
| 160 | 15 | 2 | 1 | 6 | 76 | 1096 | 1.8 |

### 6.6. One-Stage Preparation of Polyamines from Cyanoethylated Precursor Molecules (Batchwise Mode)

Examples 5 to 10 were conducted analogously to example 1b.

### Example 5

As feedstock, 70 g of N,N′-bis(2-cyanoethyl)piperazine dissolved in 70 g of THF were initially charged under nitrogen in the pressure vessel. 50 g of the activated catalyst were fixed in a “metal cage”, through which the stirred reaction mixture flowed. The polymerization was effected at 160° C. and total pressure 50 bar for 5 hours. The reaction outputs were analyzed by gas chromatography (% by weight) and by gel permeation chromatography (absolute calibration by measurement of defined polyamine standards). The composition of the crude output is summarized in table 6.

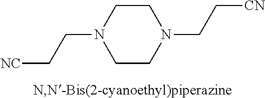 [SMILES: N#CCCN1CCN(CCC#N)CC1]

TABLE 6

| | | N-(3- | N,N′-Bis(3- | | | | |
|---|---|---|---|---|---|---|---|
| | | Aminopropyl)- | aminopropyl)- | | | | |
| T [° C.] | THF | piperazine | piperazine | Others | High boilers | Mw | PDI |
| 160 | 35 | 1 | 7 | 5 | 52 | 930 | 1.8 |

### Example 6

As feedstock, 60 g of N-(2-cyanoethyl)piperazine dissolved in 60 g of THF were initially charged under nitrogen in the pressure vessel. 50 g of the activated catalyst were fixed in a “metal cage”, through which the stirred reaction mixture flowed. The polymerization was effected at 160° C. and total pressure 50 bar for 5 hours. The reaction outputs were analyzed by gas chromatography (% by weight) and by gel permeation chromatography (absolute calibration by measurement of defined polyamine standards). The composition of the crude output is summarized in table 7.

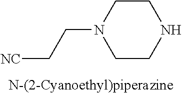

TABLE 7

| | | N-(3- | | | | | |
|---|---|---|---|---|---|---|---|
| T | | Aminopropyl)- | | | High | | |
| [° C.] | THF | piperazine | Dimer | Others | boilers | Mw | PDI |
| 160 | 32 | 10 | 19 | 7 | 32 | 400 | 1.6 |

### Example 7

As feedstock, 70 g of N,N′-bis(2-cyanoethyl)phenylene-1,3-diamine dissolved in 70 g of THF were initially charged under nitrogen in the pressure vessel. 50 g of the activated catalyst were fixed in a “metal cage”, through which the stirred reaction mixture flowed. The polymerization was effected at 160° C. and total pressure 50 bar for 5 hours. The reaction outputs were analyzed by gas chromatography (% by weight) and by gel permeation chromatography (absolute calibration by measurement of defined polyamine standards). The composition of the crude output is summarized in table 8.

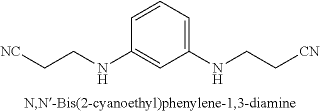 [SMILES: N#CCCNc1cccc(NCCC#N)c1]

TABLE 8

| | | N-(3- | N,N′-Bis(3- | | | | |
|---|---|---|---|---|---|---|---|
| | | aminopropyl)- | aminopropyl)- | | | | |
| | | phenylene-1,3- | phenylene-1,3- | | | | |
| T [° C.] | THF | diamine | diamine | Others | High boilers | Mw | PDI |
| 160 | 31 | 12 | 8 | 9 | 40 | 440 | 1.8 |

### Example 8

As feedstock, 70 g of N,N′-bis(2-cyanoethyl)tolylene-2,4-diamine dissolved in 70 g of THF were initially charged under nitrogen in the pressure vessel. 50 g of the activated catalyst were fixed in a “metal cage”, through which the stirred reaction mixture flowed. The polymerization was effected at 160° C. and total pressure 50 bar for 5 hours. The reaction outputs were analyzed by gas chromatography (% by weight) and by gel permeation chromatography (absolute calibration by measurement of defined polyamine standards). The composition of the crude output is summarized in table 9.

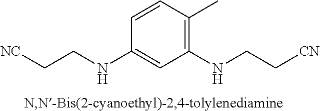 [SMILES: Cc1ccc(NCCC#N)cc1NCCC#N]

TABLE 9

| | | N,N′-Bis(3- | | | | |
|---|---|---|---|---|---|---|
| | | aminopropyl)-2,4- | | | | |
| T [° C.] | THF | tolylenediamine | Others | High boilers | Mw | PDI |
| 160 | 30 | 28 | 22 | 20 | 350 | 1.7 |

### Example 9

As feedstock, 70 g of N,N′-bis(2-cyanoethyl)aniline dissolved in 70 g of THF were initially charged under nitrogen in the pressure vessel. 50 g of the activated catalyst were fixed in a “metal cage”, through which the stirred reaction mixture flowed. The polymerization was effected at 160° C. and total pressure 50 bar for 5 hours. The reaction outputs were analyzed by gas chromatography (% by weight) and by gel permeation chromatography (absolute calibration by measurement of defined polyamine standards). The composition of the crude output is summarized in table 10.

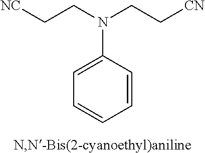 [SMILES: N#CCCN(CCC#N)c1ccccc1]

TABLE 10

| | | N,N′-Bis(3- | | | | |
|---|---|---|---|---|---|---|
| | | aminopropyl)- | | | | |
| T [° C.] | THF | aniline | Others | High boilers | Mw | PDI |
| 160 | 35 | 33 | 27 | 5 | 1097 | 2.6 |

### Example 10

As feedstock, 70 g of N,N′-bis(2-cyanoethyl)methyldiaminecyclohexane dissolved in 70 g of THF were initially charged under nitrogen in the pressure vessel. 50 g of the activated catalyst were fixed in a “metal cage”, through which the stirred reaction mixture flowed. The polymerization was effected at 160° C. and total pressure 50 bar for 5 hours. The reaction outputs were analyzed by gas chromatography (% by weight) and by gel permeation chromatography (absolute calibration by measurement of defined polyamine standards). The composition of the crude output is summarized in table 11.

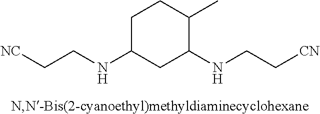 [SMILES: CC1CCC(NCCC#N)CC1NCCC#N]

TABLE 11

| | | N,N′- | | | | |
|---|---|---|---|---|---|---|
| | | Bis(3-aminopropyl)- | | | | |
| | | methyl- | | High | | |
| T [° C.] | THF | diaminocyclohexane | Others | boilers | Mw | PDI |
| 160 | 31 | 33 | 16 | 53 | 593 | 1.7 |

1: A process for preparing polyamines, the process comprising converting compounds of formula (I)
NC—Zₘ—(X)ₙ—Y (I)
where
Z is CH₂—CH₂—NR—,
m=0 or 1,
where in the case that m=0
NC— is bonded directly to X,
in the case that m=1 and n=1
NR is bonded to X and R=H or CH₂—CH₂—CN, and
in the case that m=1 and n=0
either R or NR is selected from the group consisting of CH₃, a phenyl group, CH₂—CH₂—CN and CH₂—CH₂—CH₂—NH₂ or
the nitrogen of the NR is part of a heterocycle selected from the group consisting of a piperazine, a (benz)imidazole, a pyrazole, a triazole, and a diazepane, in which another nitrogen of the heterocycle is bonded to Y,
X is selected from the group consisting of an alkyl group-substituted or unsubstituted methylene, ethylene, propylene, butylene, pentylene, hexylene, heptylene, octylene, nonanylene, decanylene, undecanylene, or dodecanylene group; an alkyl group-substituted or unsubstituted 1,2-, 1,3- or 1,4-benzyl group; a 1,4′-bicarbonyl group; a 9,10-anthracenyl group; an alkyl group-substituted or unsubstituted 1,2-, 1,3- or 1,4-cycloalkyl group; and a substituted or unsubstituted phenyl-N group of a piperazine, (benz)imidazole, pyrazole, triazole, or diazepane, where the bond to CN or Z and Y or H in each case is via the two nitrogen atoms of the ring, one to two methylene groups may be substituted by linear or branched C₁- to C₁₂-alkyl, C₆- to C₁₀-aryl, C₇- to C₁₂-aralkyl, or C₃- to C₈-cycloalkyl, and at least one methylene group may be replaced by O, NH or NR, where R has the same definition as above and C₁- to C₁₂-alkyl, C₆- to C₁₀-aryl, C₇ to C₁₂-aralkyl, C₃- to C₈-cycloalkyl groups may in turn be substituted by alkyl groups,
n is 0 or a natural number from 1 to 10, and
Y in the case that m=0 and n=1 or higher
is CN, CH₂—NH₂ or CH₂—CH₂—CN,
in the case that m=1 and n=1 or higher
is NH₂ or NR—CH₂—CH₂—CN,
in the case that m=1 and n=0
is H, CH₂—CH₂—CN, CH₂—CH₂—CH₂—NH₂, alkyl group-substituted or unsubstituted benzyl or cycloalkyl group, and
in the case that m=0 and n=0
is CH₂—CN, CH₂—CH₂—CN, CH₂—CH₂—CH₂—CN, CH₂—CH₂—CH₂—CH₂—CN, CH₂—CH₂—CH₂—CH₂—CH₂—CN, CH₂—NH₂, CH₂—CH₂—NH₂, or CH₂—CH₂—CH₂—NH₂,
in one stage at temperatures in a range f from 100 to 200° C. and pressures in a range of from 20 to 200 bar, in the presence of hydrogen and a heterogeneous catalyst comprising one or more elements of transition groups 8-10 of the Periodic Table, and in one or two or more reactors, to obtain the polyamines,
wherein if the process is conducted in two or more reactors, then the heterogeneous catalyst is the same for each of the two or more reactors.

2: The process of claim 1, wherein the entire process is conducted in one reactor.

3: The process of claim 1, wherein each reaction stage of the process is conducted at the same pressure and the same temperature.

4: The process of claim 1, wherein the compounds of formula I are dinitriles of formula Ia
NC—(—X—)ₙ—CN (Ia)
or formula IIa
NC—CH₂—CH₂—NR—(—X—)ₙ—NH—CH₂—CH₂—CN (IIa)
where R, n, and X are as defined in claim 1.

5: The process of claim 1, wherein the compounds of formula I are amino nitriles of formula Ib
NC—(—X)ₙ—CH₂NH₂, (Ib)
or formula Ib′
NC—(—X)ₙ (Ib′)
or formula IIb
NC—CH₂—CH₂—NR—(—X)ₙ—NH₃ (IIb)
where R, n, and X are as defined in claim 1.

6: The process of claim 1, wherein the one or more elements of the heterogeneous catalyst is/are selected from the group consisting of Co, Ni, and Cu.

7: The process of claim 1, wherein the heterogeneous catalyst is an unsupported Co catalyst comprising up to 99% by weight of Co.

8: The process of claim 1, wherein the compounds of formula I are dinitriles selected from the group consisting of malononitrile, succinonitrile, glutaronitrile, adiponitrile, suberonitrile, 2-methylmalononitrile, 2-methylsuccinonitrile, 2-methylglutaronitrile, 1,2-, 1,3- and 1,4-dicyanobenzene, 9,10-anthracenodicarbonitrile, and 4,4′-biphenyldicarbonitrile.

9: The process of claim 1, wherein the compounds of formula I are amino nitriles used-ee selected from the group consisting of 6-aminocapronitrile, 4′-aminobutyronitrile, 3′-aminopropionitrile and 2′-aminoacetonitrile.

10: The process of claim 1, wherein an excess of hydrogen, based on the amount of hydrogen theoretically needed for hydrogenation of the nitrile groups, of 1 to 5 moles is introduced into a liquid or gas phase of a first reactor, and excess hydrogen together with ammonia formed in the converting is removed from a last reactor.

11: The process of claim 1, wherein ammonia is separated from a discharged ammonia/hydrogen mixture and discharged from the process, and hydrogen separated off is wholly or partly recycled into a reactor.

12: The process of claim 1, wherein an ammonia/hydrogen gas mixture is separated by distillation or after cooling by phase separation.

13: The process of claim 1, wherein a portion of ammonia separated off is recycled into a first reactor.

14: The process of claim 1, which is conducted batchwise or continuously.

15: The process of claim 1, wherein the one or two or more reactors reactor is/are tubular reactors or shell and tube reactors.

16: The process of claim 1, wherein unconverted nitriles and amino nitriles from a crude polyamine output and diamines and polyamine oligomers formed therefrom are separated off by distillation and recycled into a first reactor.
